(19) United States
(12) Patent Application Publication
(10) Pub. No.: US 2023/0232798 A1
(43) Pub. Date: Jul. 27, 2023

(54) DEVICE FOR SORTING INSECTS FOR SORTING A MIXTURE COMPRISING NYMPHS

(71) Applicant: Ynsect, Évry-Courcouronnes Cedex (FR)

(72) Inventors: Thibault SARTON DU JONCHAY, Chevrières (FR); Pedro ESCALANTE, Dolores Alicante (ES); Arturo ESCAROZ CETINA, Eindhoven (NL); Mathieu CHÂTEAU, Evry-Courcouronnes (FR); Solène COMPARAT, Vasselin (FR)

(73) Assignee: Ynsect, Évry-Courcouronnes Cedex (FR)

(21) Appl. No.: 18/009,531

(22) PCT Filed: Jun. 15, 2021
(86) PCT No.: PCT/FR2021/051070

§ 371 Date: Dec. 9, 2022

(30) Foreign Application Priority Data
Jun. 18, 2020 (FR) 2006379

(51) Int. Cl. A01K 67/033; B07B 1/08

(52) U.S. Cl. CPC A01K 67/033; B07B 1/08

(57) ABSTRACT

The invention relates to a method of sorting insects comprising the successive steps of:
providing (S1) a mixture comprising nymphs, and
sieving (S3) enabling separation of the nymphs from the rest of the mixture comprising sieving said mixture with a sieve having a screen of plastic material (S4).

This method may be complemented by densimetric sorting.

The invention also relates to a corresponding insect sorting device.

The present invention relates to the field of sorting insects, for example in particular in the context of insect rearing.

The insects concerned by the invention are crawling insects, or insects that are primarily crawling insects, or flying insects at stages of their development at which they do not fly (larvae, nymphs). The insects concerned by the invention are thus for example the Coleoptera, Diptera, Lepidoptera, Isoptera, Orthoptera, Hymenoptera, Blattoptera, Hemiptera, Heteroptera, Ephemeroptera and Mecoptera, preferably, Coleoptera, Diptera, Orthoptera, Lepidoptera.

The invention finds a preferential application in the sorting of yellow mealworms, also called Tenebrio molitor.

Unless otherwise specified, the term “insect” is used to designate any stage of development from the egg or ootheca to the adult insect.

The production of insects on a large scale has numerous advantages, in particular in the agro-industry. Some edible insect species are indeed rich in protein and may in particular serve for feeding animals, for fish, crustaceans, and some fowl. Large scale insect rearing also has advantages in other industrial sectors. For example, the exoskeleton of insects is in large part constituted by chitin, of which a known derivative is chitosan. The applications for chitin and/or chitosan are numerous: cosmetic (cosmetic composition), medical and pharmaceutical (pharmaceutical composition, treatment of burns, biomaterials, corneal plasters, surgical thread), dietary and food, technical (filtering agent, texturizer, flocculent or adsorbent for example in particular for filtering and decontaminating water), etc.

Document FR3034622 presents a farm configured to large-scale insect rearing, that is to say at industrial scale. The rearing therein employs rearing containers (typically tubs) which are stacked to form basic rearing units. The basic rearing units are stored in a first zone, and, when a rearing operation is to be carried out, the containers are brought to a station configured for performing the operation, grouped into basic rearing units or ungrouped on a unitary basis.

The rearing operations concern non-exhaustively feeding, provision of water, the grading of the insects, the addition of insects to rearing containers, and numerous and various sorting operations which, during rearing, enable the insects to be separated or classified according to their stage of development, or to separate live insects from dead insects and/or from their rearing medium, etc.

Numerous sorting steps are thus carried out during rearing. These sorting steps are complex to perform at large scale, since the sorting methods generally envisioned are manual methods which do not enable efficient and fast sorting.

Certain solutions have been envisioned in the state of the art, in order to automate certain sorting operations. These sorting operations are often based on the dimensions of the components to sort, using successive sieves or sifters for this. For example, document US8025027 discloses a complex sieve system with four levels of separation, provided for the sorting of insects.

However, the solutions proposed are imperfect, in particular for sorting operations during which fragile insects are sorted. This is for example in particular the case for the yellow mealworm, when a sorting operation concerns a mixture, or a mass of product, comprising nymphs. The nymphs of the yellow mealworm are very fragile in relation to mechanical shocks. Automated sorting operations, such as envisioned in the state of the art, induce injuries and high mortality of nymphs.

The invention is thus directed to providing a method and a device, that are automated and optimized, for the sorting of insects, in particular enabling the separation of nymphs from a mixture comprising nymphs.

Thus, the invention relates to a method of sorting insects comprising the successive steps of:
providing a mixture comprising nymphs, and
sieving enabling separation of the nymphs from the rest of the mixture, said sieving step comprising sieving the mixture with a sieve having a screen of plastic material.

The sieving of the mixture with a sieve having a screen of plastic material may in particular be sieving with a polyurethane screen sieve.

The applicant has discovered that sieving with a screen of plastic material, uncommon in industry and unknown in the state of the art in the field of insect rearing, makes it possible to greatly reduce the damage caused to fragile insects by sieving, particularly for example to insect nymphs, and especially to the nymphs of yellow mealworms.

The sieving of the mixture with a plastic screen sieve may be carried out with a sieve having a screen inclined relative to a horizontal plane. The inclined screen is less contusion-inducing for the insects and enables effective sieving. The sieving of the mixture with a screen of plastic material may comprise imparting circular or oval movement to the screen of plastic material in its plane of extension, said movement being free of any component at a right angle to said plane of extension of the sieve. Such a movement improves the quality of the sorting and limits the shocks suffered by the insects, in particular by the nymphs. The mixture may comprise larvae, and the sieving step may furthermore enable separation of the larvae having dimensions greater than predefined dimensions.

The sieving step may comprise sieving the rest of the mixture after separating the nymphs with at least a second sieve.

It is thus possible to obtain several fractions from the initial mixture, according to the size of the components constituting it. It is notable that the nymphs, which are insects at the stage of development at which they are the most fragile, only undergo a single sorting operation, that is to say a single sieving operation.

The sieving provided also enables the collection of the largest larvae, which may advantageously be employed in the rearing to obtain the adult insects which will serve for reproduction.

The method may furthermore comprise, after the sieving step, a step of densimetric separation of at least some of the rest of the mixture after the separation of the nymphs. Said at least some of said rest of the mixture may comprise larvae, and the step of densimetric separation may be configured to separate the larvae from said at least some of said rest of the mixture.

The step of densimetric separation may comprise the passage of the at least some of said rest of the mixture after the separation of the nymphs in at least one densimetric separator selected from: a densimetric table, a dust separator, and a densimetric column.

In certain embodiments, the invention thus makes it possible, in addition to the separation of the nymphs, to harvest the larvae for producing a larvae-based product (for example for feeding animals, cosmetics, etc.). The nymphs, separated in advance, are not subjected to the densimetric separation, which is liable to damage them.

Among the separators envisioned, the dust separator is particularly advantageous in that it provides sufficient effectiveness, low bulk, and enables the sorting of a large amount of mixture in a short time.

The invention also relates to an insect sorting device, comprising
a feeder enabling the supply of a mixture containing nymphs,
a sieving device comprising a sieving screen of plastic material,
a densimetric separator, the insect sorting device being configured such that only at least some of the mixture passing through the sieving screen passes into the densimetric separator.

In such a device, the sieving screen may be of polyurethane. The sieving screen may be inclined relative to a horizontal plane. The sieving device may comprise at least one second sieving screen.

The densimetric separator may comprise at least one apparatus selected from: a densimetric table, a dust separator, and a densimetric column. Still other features and advantages of the invention will appear in the following description.

In the accompanying drawings, given by way of non-limiting example:

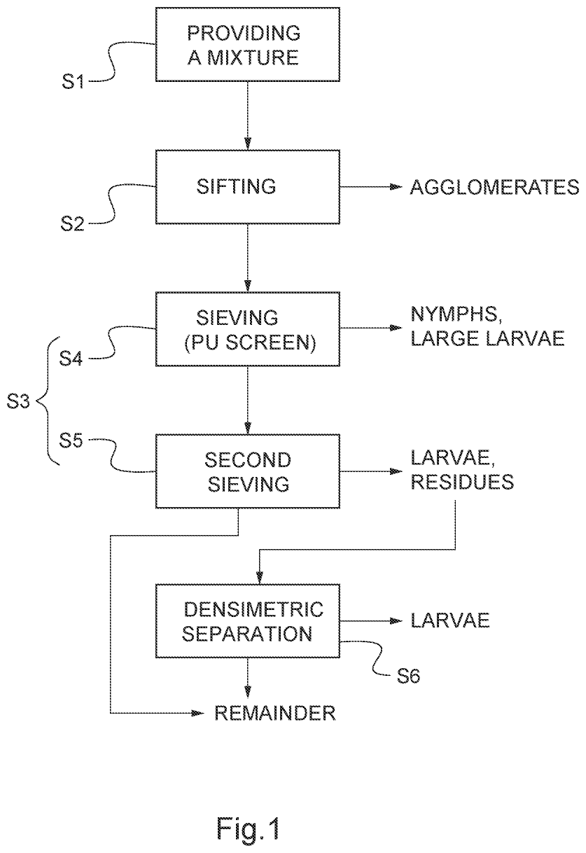

FIG. 1 shows a flowchart of a method in accordance with an embodiment of the invention;

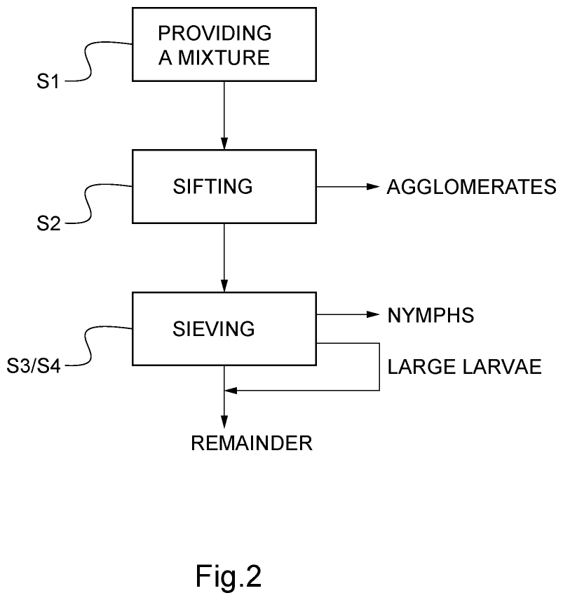

FIG. 2 shows a flowchart of another method in accordance with an embodiment of the invention;

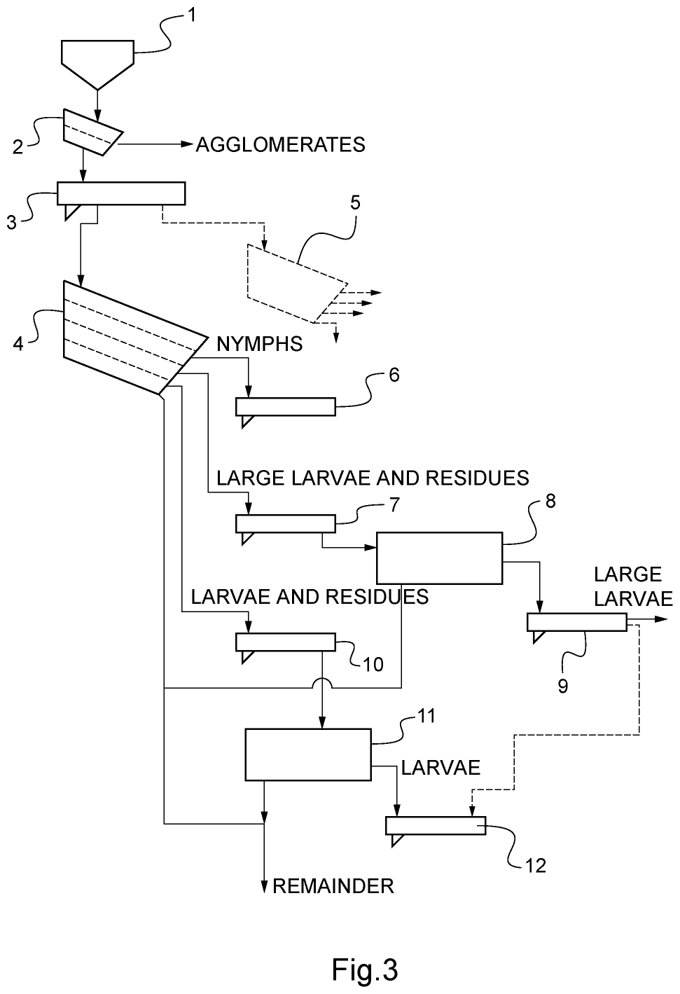

FIG. 3 is a schematic representation of a sorting device in accordance with an embodiment of the invention;

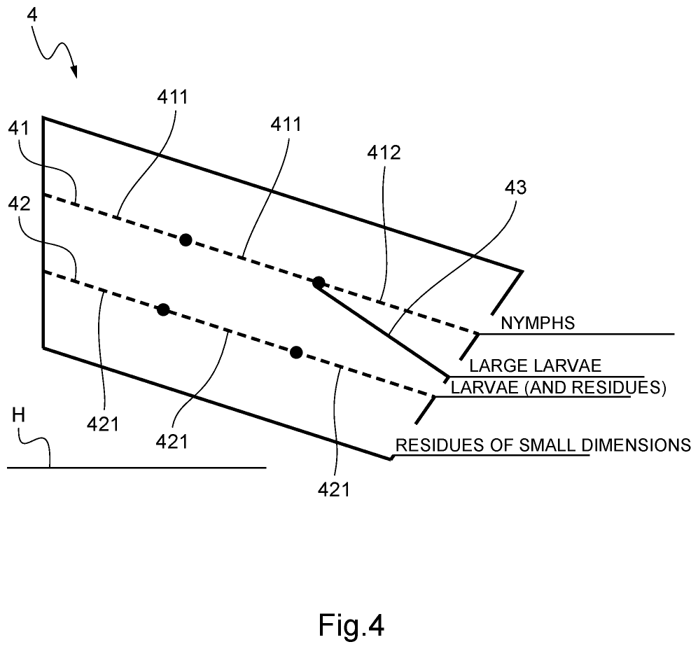

FIG. 4 is a schematic representation of a first aspect of the sorting device of FIG. 3;

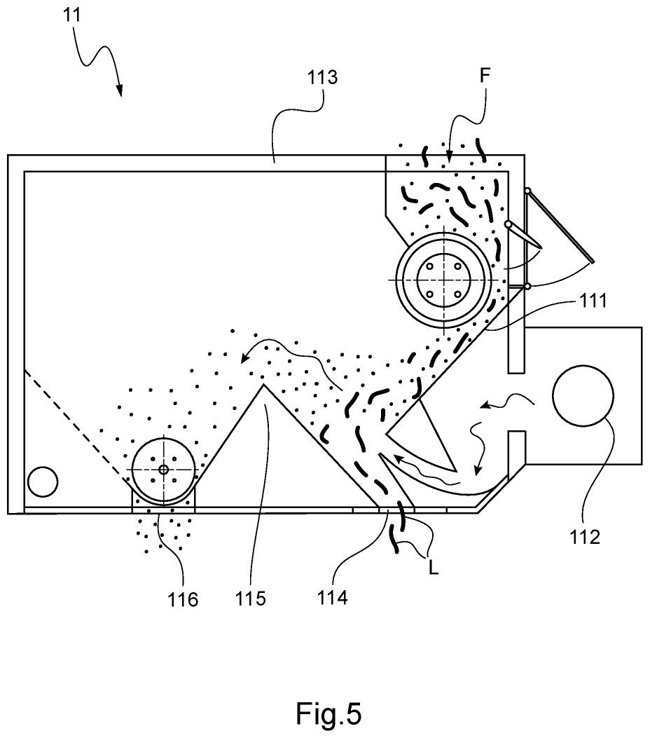

FIG. 5 is a schematic representation of a second aspect of the sorting device of FIG. 3.

FIG. 1 shows a flowchart of a method in accordance with an embodiment of the invention. This method makes it possible to separate the nymphs and, possibly, the larvae, of a mixture comprising insects at these stages of development from the other components contained in the mixture, such as the rising medium, dejections, dead insects, etc.

In general, in the present document, and unless otherwise indicated, the term “larva” designates a living larva.

In a step S1 of providing a mixture, such as mixture is supplied, for example by collection and emptying of the rearing tubs.

This mixture is, in the example shown and which comprises a sifting step S2, passed into a sifter having openings of large size, of typically 1 cm or more, for example in the form of square mesh having openings with 1 cm sides. These agglomerates constitute solid rearing substrate (material in which are raised the insects and which may contain their food, if present) which has combined to form compact masses, the dimensions of which are generally comprised between 1 cm and 10 cm.

The mixture, freed of the agglomerates, is next sieved in a sieving step S3.

In the example shown, two successive sieving operations are carried out in the sieving step S3, i.e. a sieving operation S4 with a screen of plastic material and a sieving operation S5 with a second sieve. In practice, these two or more sieving operations may be carried out in a single sieving device comprising several sieves and/or several sieving stages or “decks”.

The sieving step S3 thus comprises first of all a sieving operation by a sieve comprising a screen of plastic material, for example and advantageously of polyurethane (PU). The use of this material, compared with the screens of metal mesh used more conventionally, has proved particularly advantageous.

By “screen” is meant the filtering part of the sieve, that is to say the network of open mesh formed within the frame of the sieve.

In particular, the use of a sieve screen of plastic, makes it possible to avoid or limit the injuries to the insects contained in the mixture during sieving. In particular, the insect nymphs, for example especially yellow mealworm, are very fragile and capable of being damaged by shocks suffered during sieving. A plastic screen, particularly of polyurethane, has proved less contusion-inducing for the insects, in particular for the nymphs.

The applicant has furthermore noted that the use of a sieve of which the movements are substantially made in the plane of the screen of the sieve, without or substantially without any component at a right angle to the plane of the screen of the sieve, also makes it possible, in addition to the use of a screen of plastic material, to limit or even eliminate damage to the nymphs and their mortality on sorting. It has furthermore been noted that a circular or oval movement of the sieve in its plane of extension appreciably improves the sorting.

The sieving S4 with a screen of plastic material thus makes it possible to separate the nymphs, and optionally the largest larvae, from the rest of the mixture.

The nymphs of yellow mealworm have an elongate shape, having a length of 14 mm to 20 mm and transverse dimensions included within a circle of the order of 5 mm to 6 mm. The largest larvae may for example be defined as being the larvae weighing more than 125 mg, which corresponds to the larvae measuring more than 25 mm in length and having transverse dimensions included within a circle of the order of 3 mm to 4 mm.

As is detailed with reference to FIG. 3, it is possible with the same sieve deck, that enables the nymphs and the largest larvae to be separated from the rest of the mixture, also, optionally, to separate the nymphs from the largest larvae. This separation (between nymphs and the largest larvae) may alternatively be carried out in a separate manual or automated sorting step (for example with another plastic screen sieving device.

The sieving with a second sieve S5 for example makes it possible to separate the larvae of smallest size from part of the rest of the mixture. Although the importance of using a sieve of which the screen is of plastic material is not so great for this sieving operation, the larvae, which correspond to the development stage at which the insects are the most fragile, having been separated in the preceding sieving operation, the use of a screen of plastic material is nevertheless preferable for this second sieving operation (sieving S5 with a second sieve).

Further to the sieving step, there are obtained separately:
nymphs and larvae of large size (together or separately),
a combination comprising living larvae and residues, primarily comprising dead larvae and a small amount of remaining rearing substrate and/or insect dejections, referred to as “frass”),
the rest of the initial mixture, which contains no more or practically no more living insects (nymphs or larvae).

In the method illustrated in FIG. 1, the combination comprising larvae and residues is next subjected to densimetric separation S6. Thus, in this example embodiment comprising two sieving operations, only part of the rest of the mixture after separation of the nymphs is subjected to the densimetric separation S6.

Importantly, in the method of the invention, the nymphs initially present in the mixture are separated and are not subjected to the densimetric sorting which would be liable to damage them.

The densimetric separation makes it possible to eliminate residue from the mixture containing larvae (living) which results from the sieving step S3. The densimetric separation S6 thus enables the separation of this part of the mixture into two fractions, according to their density. The living larvae, more dense, are thus separated from the rest of the mixture, which consists of less dense components, such as dead larvae, insect dejections if present (referred to as “frass”), and the rearing substrate which is for example primarily wheat bran.

If need be, in certain embodiments, the large larvae coming from the sieving may also be subjected to densimetric separation in order to eliminate possible residues, including dead insects (or dead insect parts) which could have passed with them at the time of sieving (for example components of the rearing substrate onto which the larvae had gripped on sieving).

The method of FIG. 1 corresponds to a sorting method making it possible to separate the nymphs, the larvae of large size, and the smallest larvae from their rearing medium (substrate, dejections, dead insects).

The nymphs and the larvae of large size (which are closer in their development cycle to the adult stage than the smallest larvae) may for example be provided for the reproduction of the insects.

The larvae of smallest size, and, possibly, some of the larvae of large size may for example be sent for killing for producing products of high added value (for feeding animals, the chemical industry, cosmetics, etc.).

The invention may, according to some embodiments, be employed for other sorting operations to be carried out during insect rearing. Another example is thus shown in FIG. 2.

For example, when rearing yellow mealworms, it is necessary to separate the nymphs from the larvae to isolate the nymphs from the larvae at the time of their metamorphosis. This avoids cannibalism by the larvae of the nymphs.

Thus, such a sorting method comprises the steps presented above of providing a mixture S1, the mixture containing nymphs, and optionally sifting S2. The method continues with the sieving step S3.

For this sorting operation, the sieving S3 solely comprises sieving S4 with a screen of plastic material, advantageously of polyurethane, making it possible to separate the nymphs from the rest of the mixture. The large larvae collected during the sieving are separated from the nymphs and sent back with the rest of the mixture for continuation of the rearing.

FIG. 3 shows an insect sorting device according to an embodiment of the invention, which is configured for performing the sorting method of FIG. 1.

A hopper, constituting a feeder 1, makes it possible to receive and supply the mixture containing insects, in particular nymphs. The mixture is passed into a sifter 2, which makes it possible to separate the agglomerates from the rest of the mixture.

The remaining mixture, coming out from the sifter 2, is deposited on a first conveyor 3. The first conveyor 3 (for example a belt conveyor) makes it possible to convey and distribute the mixture into one or more sieving devices 4, 5.

The sieving device 4 makes it possible to separate the mixture that is introduced therein into four fractions, as will be detailed below with reference to FIG. 4.

The following fractions of the mixture introduced into the sieving device are thus separated:
the nymphs,
the larvae of large size (“large larvae”), possibly with residue.
the larvae of smallest size, with residue, in particular dead larvae;
the components of very small size, which may correspond to part of the rearing substrate, dust, frass, etc.

The nymphs are deposited onto a second conveyor 6, for example to be disposed in rearing tubs where they will continue their metamorphosis.

The larvae of large size are deposited on a third conveyor 7. They are for example provided to be reared to reach the adult stage, for the reproduction of the insects in the farm. In the example embodiment represented here, the fraction output from the sieving device comprising the larvae of large size also comprises residues, which are separated from the larvae of large size by an optional densimetric separator 8. On exiting the optional densimetric separator 8, the larvae of large size are freed from residue, in particular dead larvae, and deposited on a fourth conveyor 9. Some of these larvae may thus be sent for killing for the production of a product.

The fraction of the mixture comprising the larvae of smallest size with residues is deposited on a fifth conveyor 10, which transports it to a densimetric separator 11. The densimetric separator makes it possible to separate the larvae from the residue, in particular from the dead insects. The densimetric separator may be produced according to various technologies enabling sorting based on the density of the components constituting the mixture sorted therein. Thus, the larvae, which are denser than the residues, are separated from said residues. The densimetric separator may in particular be selected from a densimetric table, a dust separator, and a densimetric column, for example cyclone-based.

A dust separator, which constitutes a particularly advantageous densimetric separator in the context of the present invention, is diagrammatically illustrated and explained below with reference to FIG. 5.

The larvae freed from residues are deposited on a sixth conveyor 12, to be conducted for killing for the production of a product based on insect larvae.

The rest of the mixture, which is formed from components of very small size coming from the sieving device or devices 4, from residues coming from the densimetric separator 11, from the optional densimetric separator 8, if present, and, if present, from the agglomerates coming from the sifter, is collected for it to be treated.

If the device comprises several sieving devices 4, 5, the apparatuses following the sieving devices may be duplicated (several densimetric separators, etc.) or mutualized (a single densimetric separator to treat the fractions coming from several sieving devices) according to their capacity.

FIG. 4 is a diagrammatic representation of a sieving device 4 which may advantageously be employed in the invention. The sieving device 4 represented here is a two-deck sieving device, that is to say it comprises two levels of sieving, that is to say a first deck 41 and a second deck 42. The sieve formed by each deck is inclined relative to a horizontal plane H, typically corresponding to the ground (for example the floor slab) on which the sieving device is installed.

The first deck 41 is formed by a succession of gratings or screens forming a first sieve. These screens are of plastic material, here polyurethane.

The particularity of the first deck is to have screens of different mesh dimensions, that is to say one or more first screens 411 having mesh with a first aperture, and one or more second screens 412 having mesh with a second aperture.

The sieving device is in particular configured for rearing yellow mealworms. For example, the first screen or screens 411 may have square mesh, of which the aperture sides are of the order of 3.2 mm. This mesh enables the passage of the insect rearing substrate (for example wheat bran), dejections, and larvae of less than 25 mm in length which have transverse dimensions included within a circle of less than approximately 3 mm.

The large insects (larvae of large size, with transverse dimensions included within a circle of the order of 3 mm to 4 mm and constituting larvae of more than 25 in length, and a mass of more than 125 mg) as well as the nymphs cannot pass through that first screen or screens 411.

That second screen or screens 412 in this example have square mesh, of which the aperture sides are of the order of 5 mm. This mesh enables the passage of larvae of large size, but not that of the nymphs, which have a length of 14 mm to 20 mm and transverse dimensions included within a circle of the order of 5 mm to 6 mm.

The larvae of large size (“large larvae”) are collected on a plane 43 interposed between the first deck 41 and the second deck 42, under the second screen or screens 412. The second deck comprises, in the example shown, third screens 421 of which the mesh is identical, that is to say square mesh of which the aperture sides are of the order of 1.5 mm. This mesh enables the passage of certain residue of small size, but not that of the larvae, and generally not that of the dejections.

The sieving device 4 with two decks shown in FIG. 4 thus enables the collection of four separate fractions of the initial mixture.

Of course, the dimensions mentioned above by way of example are to be adapted according to the sorting performed, the desired result, and the insect species sorted. For example, for the sorting shown in FIG. 2, the sieving device 4 may be similar, but have one or more second screens 412 of the first deck 41 having square mesh with aperture sides of the order of 15 mm.

FIG. 5 shows an example of a densimetric separator 11 which may be employed in the invention. The type of apparatus shown in FIG. 5 is generally designated by the term “dust separator”.

A fraction F of the initial mixture coming from the sieving device 4, which contains larvae and residues among which are in particular dead insects and, possibly, insect dejections is introduced into the dust separator. The fraction F is distributed over an inclined plane 111 on which it descends in a thin layer. A fan 112 produces an air stream introduced into the body 113 of the dust separator. The air stream encounters the layer formed by the fraction F, and takes with it the least dense components present in the fraction F, while the less dense components fall by gravity. The denser components, i.e. the (living) larvae L, thus leave by a first exit 114 from the dust separator. The inside volume of the dust separator may comprise a certain number of projections enabling effective separation to be provided. In particular, in the example shown, a central projection 115 is interposed between the first exit 114 of the dust separator and a second exit 116.

Thus the less dense components of the fraction F (dead larvae, other residues), which are taken by the air stream must pass over the central projection 115 before reaching the second exit 116. This leaves the time necessary for the proper separation of the fraction F, while the air stream climbing said central projection 115 prevents any exit by the first exit 114 of components of too low density.

The invention thus developed makes it possible, by an appropriate selection and adaptation of sorting machines, to sort insects in particular fragile insects in an effective and automated manner.

The invention is in particular especially well-adapted to the sorting of a mixture comprising insect nymphs, for example nymphs of yellow mealworms, which are particularly sensitive to shocks. It thus enables the collection of the nymphs without damaging them, by the use of a sieving device with a screen of plastic material, preferably inclined. In some embodiments of the invention, densimetric sorting is also carried out, without the nymphs being subjected to that densimetric sorting. The order in which are carried out the different steps of the sorting is thus fundamental. The invention thus provides a sorting method and device enabling the optimization of the sorting operations at an industrial scale.

1. Method of sorting insects comprising the successive steps of:
providing (S1) a mixture comprising nymphs, and
sieving (S3) enabling separation of the nymphs from the rest of the mixture, said sieving step (S3) comprising sieving the mixture with a sieve having a screen of plastic material (S4).

2. Method of sorting insects according to claim 1, wherein the sieving of the mixture with a sieve having a screen of plastic material (S4) is sieving with a polyurethane screen sieve.

3. Method of sorting insects according to claim 1, wherein the sieving of the mixture with a plastic screen sieve (S4) is carried out with a sieve having a screen inclined relative to a horizontal plane.

4. Method of sorting insects according to claim 1, wherein the sieving of the mixture with a screen of plastic material (S4) comprises imparting circular or oval movement to the screen of plastic material in its plane of extension, said movement being free of any component at a right angle to said plane of extension of the sieve.

5. Method of sorting insects according to claim 1, wherein the mixture comprises larvae, and wherein the sieving step (S3) furthermore enables separation of the larvae having dimensions greater than predefined dimensions.

6. Method of sorting insects according to claim 1, wherein the sieving step (S3) comprises sieving of the rest of the mixture after separating the nymphs with at least a second sieve.

7. Method of sorting insects according to claim 1, the method further comprising, after the sieving step (S3), a step of densimetric separation (S6) of at least some of the rest of the mixture after the separation of the nymphs.

8. Method of sorting insects according to claim 7, wherein the at least some of said rest of the mixture comprises larvae, and wherein the step of densimetric separation (S6) is configured to separate the larvae from the at least some of said rest of the mixture.

9. Method of sorting insects according to claim 7, wherein the step of densimetric separation (S6) comprises the passage of the at least some of said rest of the mixture after the separation of the nymphs in at least one densimetric separator (11) selected from: a densimetric table, a dust separator, and a densimetric column.

10. Device for sorting insects comprising
a feeder (1) enabling the supply of a mixture containing nymphs,
a sieving device (4) comprising a sieving screen (411, 412) of plastic material
a densimetric separator (11),
the insect sorting device being configured such that only at least some of the mixture passing through the sieving screen passes into the densimetric separator.

11. Device for sorting insects according to claim 10, wherein the sieving screen is of polyurethane.

12. Device for sorting insects according to claim 10, wherein the sieving screen (411, 412) is inclined relative to a horizontal plane (H).

13. Device for sorting insects according to claim 10, wherein the sieving device comprises at least one second sieving screen (421).

14. Device for sorting insects according to claim 10, wherein the densimetric separator comprises at least one apparatus selected from: a densimetric table, a dust separator, and a densimetric column.
